(19) United States
(12) Patent Application Publication
(10) Pub. No.: US 2026/0049885 A1
(43) Pub. Date: Feb. 19, 2026

(54) MEDICAL DEVICE

(71) Applicant: Roche Diabetes Care, Inc., Indianapolis, IN (US)

(72) Inventor: Frank Deck, Niederkirchen (DE)

(21) Appl. No.: 19/368,554

(22) Filed: Oct. 24, 2025

Related U.S. Application Data
(63) Continuation of application No. PCT/EP2024/060981, filed on Apr. 23, 2024.

(30) Foreign Application Priority Data
Apr. 26, 2023 (EP) 23 170 081.6

(51) Int. Cl. G01M 3/26; A61B 5/145; A61B 5/1473; A61B 5/1495; A61M 5/158; G01L 11/02

(52) U.S. Cl. CPC G01M 3/26; A61B 5/14532; A61B 5/1473; A61B 5/1495; A61M 5/158; G01L 11/02; A61M 2005/1588; A61M 2205/3331; A61M 2205/70; A61M 2209/02

(57) ABSTRACT

A medical device having at least one invasive portion is disclosed. The medical device has a housing at least partially surrounding a tight chamber. The tight chamber sustains a pressure difference between an interior lumen of the tight chamber and the surrounding environment. The housing also has at least one deformable component. The housing is configured such that a state of deformation of the deformable component provides for a measurable indication of the pressure difference between the interior lumen of the tight chamber and the surrounding environment.

### RELATED APPLICATIONS

This application is a continuation of International Application No. PCT/EP2024/060981, filed Apr. 23, 2024, which claims priority to EP 23 170 081.6, filed Apr. 26, 2023, the entire disclosures of both of which are hereby incorporated herein by reference.

### BACKGROUND

This disclosure relates to a medical device, a kit and a method for monitoring the integrity of a housing of a medical device. The medical device may be applied in the field of continuous monitoring of an analyte in a body fluid of a user, specifically in the field of home care and in the field of professional care, such as in hospitals. Other applications, however, are also feasible.

Medical devices that are to be used sterile on patients commonly require sterility testing during manufacturing. Direct sterility testing is usually destructive. Thereby, the medical device is commonly removed from its sterile packaging and is examined for sterility. This method is commonly of limited suitability for quality control in production as tested products can no longer be sold.

Alternatively, validated manufacturing processes and applied materials of known quality may be used. With such methods, all manufacturing parameters that can influence sterility are commonly evaluated and monitored. Products manufactured using tested parameters are expected to have the same sterile properties as samples from a single destructive test. However, if the products are transported or subjected to other stressful influences after manufacturing, the integrity of the packaging can no longer be assumed to be 100%.

As an alternative, an indirect method may be applied in which the sterility is not checked directly, but a protective mechanism that maintains sterility is checked. A sterilized product that is received in an intact packaging can still be considered sterile.

An established method for determining an integrity of a sterile container works on the principle of volume determination based on a pressure test according to Boyle-Mariotte's law. Thereby, an unknown volume V₂ can be determined with the aid of a known volume V₁ and a known initial pressure. If the volume V₂ in a test set-up is designed as a test chamber with a known, constant volume, the additional volume of an inserted test specimen can be determined. The known volume V₂ of the test chamber is reduced by the volume of the test specimen. Provided that the volume of the test specimen is also known (e.g., determined from samples), a tight test specimen can be distinguished from a leaky test specimen. In a leaky specimen, an inner volume of its sterile chamber is accessible to gas pressure, and does not reduce the volume V₂. A tight test specimen reduces the volume V₂ not only by its solid content, but additionally by the tightly sealed volume inside the sterile chamber. This type of test is described in DIN EN 1779, among others.

According to the state of the art, test methods for leak testing of wristwatches are also known. For this purpose, the wristwatch to be tested is placed in a pressure chamber and an outer case thickness which corresponds to a distance between the watch glass and the back cover is measured. If the distance remains constant during pressurization, the housing of the wristwatch is leaking. The wristwatch acts as a pressure can whose thickness is measured.

WO 2022/147329 A1 describes a sensor control device for analyte monitoring comprising electronics housing having shell defining top surface and mount defining bottom surface of the electronics housing. Adhesive patch coupled to the bottom surface defines central opening, and includes first layer facing the mount and second layer facing skin of user. The first layer has a first aperture, the second layer has a second aperture, and the first aperture and second aperture align with the central opening along the vertical axis of the sensor control device. The first layer or second layer includes laser cut slots or laser cut holes configured for drainage of fluid or breathability of skin.

U.S. Publication No. 2022/0080678 A1 describes a method which includes assembling a sensor subassembly that includes a sensor, a sensor mount, a collar, a sharp, and a sensor cap. The method includes loading a sensor in a sensor mount; dispensing adhesive into a mount channel of the sensor mount; clamping a collar to the sensor mount; and curing the adhesive to fix the collar to the sensor mount. The method can also include inserting a sharp into the sensor mount over the sensor an attaching a sensor cap to the sensor and sensor sharp to provide a sealed sensor subassembly. Methods of assembling an on-body sensor puck assembly and an applicator assembly, and a sensor including a tail, a flag, and a neck that interconnects the tail and the flag and methods of configuring a sensor are also disclosed.

EP 2 982 383 B1 describes assembling an analyte sensor with an analyte sensor insertion device, packaging the assembled analyte sensor and sensor insertion device in a substantially airtight seal, and irradiating the packaged assembled analyte sensor and sensor insertion device at a predetermined dose using one or more electron beam accelerators.

Despite the advantages achieved by the above-mentioned devices, several technical challenges remain. According to the state of the art, the sterility of a product is verified either destructively or indirectly. If a 100 percent control is desired, destructive testing is commonly not suitable for production processes. Further, medical devices in the field of continuous monitoring of an analyte in a body fluid of a user typically comprise a sterile chamber in which an analyte sensor is located and, additionally, comprise a further enclosed volume, namely, a volume surrounded by an electronics unit housing. The electronics unit housing commonly does not have to meet the requirements of sterile tightness, but commonly has to be waterproof and dustproof. A disadvantage of the methods of integrity testing described above is an influence of this further enclosed volume, namely, the volume enclosed by the electronics unit housing, on a measurement result. A leaky electronics unit housing leads to the same measurement effect as a leaky sterile chamber for the analyte sensor. Thus, such a test result is typically not selective for one of the sterile chamber having the analyte sensor and a chamber enclosed by the electronics unit housing.

A test method based on the “pressure can” principle with a length measurement as described above is commonly not applicable for testing the integrity of the sterile chamber having the analyte sensor since the high strength of the sterile chamber in conjunction with the small surface area of the sterile chamber commonly leads to a very small deflection.

### SUMMARY

This disclosure teaches a medical device, a kit and a method for monitoring integrity of a housing of a medical device which at least partially address the above-mentioned technical challenges. Specifically, a medical device, a kit and a method for monitoring an integrity of a housing of a medical device are disclosed which allow a reliable and destructive-free monitoring of the integrity of a housing of a medical device.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “housing,” “lumen,” “chamber” and “deformation sensor,” to name just a few, should be interpreted wherever they appear in this disclosure and claims to mean “at least one” or “one or more” regardless of whether they are introduced with the expressions “at least one” or “one or more. ” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

The term “user” as used herein is a broad term and is to be given its ordinary and customary meaning to a person of ordinary skill in the art and is not to be limited to a special or customized meaning. The term specifically relates to a person intending to monitor an analyte value, such as a glucose value, in a person's body tissue and/or to deliver a medication, such as insulin, into the person's body tissue. In an embodiment, the term specifically may refer, without limitation, to a person using the medical device. However, in an embodiment, the person using the medical device is different from the user. For example, the medical device or a part of the medical device may be inserted by the person different from the user into the user's body tissue. For example, the user may be a patient suffering from a disease, such as diabetes.

The term “body fluid” as used herein is a broad term and is to be given its ordinary and customary meaning to a person of ordinary skill in the art and is not to be limited to a special or customized meaning. The term specifically relates to an arbitrary fluid which typically is present in a body or body tissue of a user or a patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of at least one analyte, the body fluid may be present within the body or body tissue.

In a first aspect of this disclosure, a medical device having at least one invasive portion is disclosed. The medical device comprises at least one housing at least partially surrounding at least one tight chamber. The tight chamber is configured for sustaining a pressure difference between at least one interior lumen of the tight chamber and a surrounding environment. The housing comprises at least one deformable component. The housing is configured such that a state of deformation of the deformable component provides for a measurable indication of the pressure difference between the interior lumen of the tight chamber and the surrounding environment.

The term “medical device” as used herein is a broad term and is to be given its ordinary and customary meaning to a person of ordinary skill in the art and is not to be limited to a special or customized meaning. The term specifically may refer, without limitation, to an arbitrary element or article being configured for use in the field of medical technology, specifically in the field of medical analytics or medical diagnostics. The medical device may be configured for performing at least one medical function and/or for being used in at least one medical process, such as one or more of a therapeutic process, a diagnostic process or another medical process.

The medical device may be configured to be mounted on a skin site of an extremity of the user. The extremity may be selected from the group consisting of: an arm, specifically an upper arm; a stomach; a shoulder; a back; hip; a leg. Specifically, the extremity may be the upper arm. However, also other applications may be feasible.

The medical device may comprise at least one component which may be configured to stay outside of the body tissue. Further, the medical device comprises, as outlined above, the at least one invasive portion. The invasive portion may configured for being inserted at least partially into the body tissue of the user.

The medical device may be selected from the group consisting of: a medication device for delivering at least one therapeutical medical fluid to the user, specifically a device for delivering insulin to the user; a device for detecting at least one analyte in the body fluid of the user, specifically a device for detecting glucose in the body fluid of the user. Specifically, the device for detecting glucose in the body fluid of the user may be a continuous glucose monitoring system. Also other embodiments of the medical device may be feasible.

As outlined above, the medical device has the at least one invasive portion. The term “invasive portion” as used herein is a broad term and is to be given its ordinary and customary meaning to a person of ordinary skill in the art and is not to be limited to a special or customized meaning. The term specifically may refer, without limitation, to an arbitrary part or component of an element configured to be at least partially transcutaneously or subcutaneously implanted, inserted and/or positioned into a body tissue of a user. The insertion of the invasive portion of the medical device may exemplarily be performed by using an insertion device. After insertion, the invasive portion of the medical device or at least a component of the invasive portion may remain in the body tissue of the user for a predetermined period of time, such as for several hours, specifically for one or more days, more specifically for up to one week, even more specifically for up to two weeks or even more.

The invasive portion may be selected from the group consisting of: at least one analyte sensor for detecting at least one analyte in the body fluid of the user; at least one insertion component; at least one infusion cannula; at least one stimulating electrode. Other embodiments may be feasible.

The analyte sensor may be configured for being used in qualitatively and/or quantitatively detecting the at least one analyte. The term “analyte” as used herein is a broad term and is to be given its ordinary and customary meaning to a person of ordinary skill in the art and is not to be limited to a special or customized meaning. The term specifically may refer, without limitation, to a chemical and/or biological substance which takes part in the metabolism of the body of the user. Specifically, the analyte may be a metabolite or a combination of two or more metabolites. As an example, the analyte may be selected from the group consisting of: glucose, lactate, triglycerides, cholesterol. Still, other analytes or combinations of two or more analytes may be detected. The term “analyte sensor” as used herein is a broad term and is to be given its ordinary and customary meaning to a person of ordinary skill in the art and is not to be limited to a special or customized meaning. The term specifically may refer, without limitation, to a sensor which is capable of qualitatively or quantitatively detecting the presence and/or the concentration of the at least one analyte. The analyte sensor may be an electrochemical analyte sensor. The analyte sensor may comprise at least two electrodes. Specifically, the analyte sensor may comprise at least one two-electrode sensor. The two-electrode sensor may comprise precisely two electrodes, such as a working electrode and at least one further electrode such as a counter electrode, in particular a working electrode and a combined counter/reference electrode. The working electrode may comprise a working electrode pad and, optionally, at least one test chemical disposed thereon. The counter electrode may comprise a counter electrode pad. Additionally and optionally, one or more redox materials may be disposed thereon. The analyte sensor may further comprise one or more leads for electrically contacting the electrodes. The leads may, during insertion or at a later point in time, be connected to one or more electronic components. Preferably, the leads may already be connected to the electronic components before insertion of the analyte sensor. Further details on the electronic components are given below in more detail.

Specifically, the analyte sensor may be a needle-shaped or a strip-shaped analyte sensor having a flexible substrate and the electrodes disposed thereon. As an example, the analyte sensor may have a total length of 5 mm to 50 mm, specifically a total length of 7 mm to 30 mm. The term “total length” within the context of this disclosure relates to the overall length of the analyte sensor which means a portion of the analyte sensor which is inserted and the portion of the analyte sensor which may stay outside of the body tissue. The analyte sensor may be inserted partially into the body tissue of the user. A portion of the analyte sensor which is inserted is commonly also called in-vivo portion, a portion of the analyte sensor which may stay outside of the body tissue is commonly also called ex vivo portion. Preferably, the in vivo portion has a length in the range from 3 mm to 12 mm. The analyte sensor may further comprise a biocompatible cover, such as a biocompatible membrane which fully or partially covers the analyte sensor and which prevents the test chemical from migrating into the body tissue and which allows for a diffusion of the body fluid and/or the analyte to the electrodes. Other embodiments of electrochemical analyte sensors, such as three-electrode sensors, may be feasible. For example, the three-electrode sensor may comprise, in addition to the working electrode and the counter electrode, a reference electrode.

The term “insertion component” as used herein is a broad term and is to be given its ordinary and customary meaning to a person of ordinary skill in the art and is not to be limited to a special or customized meaning. The term specifically may refer, without limitation, to an arbitrary element which may be insertable at least partially into a body tissue, particularly in order to deliver or to transfer a further element. The insertion component may specifically be configured for supporting an insertion of the analyte sensor. In case the medical device is the device for detecting the at least one analyte in the body fluid of the user, the medical device may comprise the analyte sensor and the insertion component. The analyte sensor may remain in the body tissue of the user for the predetermined period of time whereas the insertion component may optionally be removed from the body tissue after insertion of the analyte sensor. However, alternatively, other embodiments may be feasible wherein the analyte sensor as well as the insertion component may remain in the body tissue of the user for the predetermined period of time. The insertion component may comprise a tip or a sharp end for inserting the analyte sensor into the body tissue.

The insertion component for inserting the analyte sensor into the body tissue of the user may be or may comprise an insertion cannula or an insertion needle. The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially slotted. The analyte sensor may be received within the insertion cannula, such as within a lumen of the insertion cannula. The term “insertion needle” as used herein is a broad term and is to be given its ordinary and customary meaning to a person of ordinary skill in the art and is not to be limited to a special or customized meaning. The term specifically may refer, without limitation, to a compact needle, specifically without a slot and without any hollow parts. The analyte sensor may be received on an outer surface of the insertion needle.

The term “infusion cannula” as used herein is a broad term and is to be given its ordinary and customary meaning to a person of ordinary skill in the art and is not to be limited to a special or customized meaning. The term specifically may refer, without limitation, to a hollow tube configured for delivering and/or infusing a medication into a body tissue of a user, in particular for delivering and/or infusing insulin into the body tissue of the user.

As outlined above, the medical device comprises the at least one housing. The term “housing” as used herein is a broad term and is to be given its ordinary and customary meaning to a person of ordinary skill in the art and is not to be limited to a special or customized meaning. The term specifically may refer, without limitation, to an arbitrary element which is configured for fully or partially enclosing at least one interior space and for providing protection to the interior space, such as one or more of a mechanical protection and a protection against environmental influences such as one or more of moisture, oxygen and microbial contaminations. Also other kinds of protection may be feasible. The housing, specifically, may be or may comprise a rigid housing, such as a rigid housing made of one or more of a plastic material. The housing may specifically comprise at least one wall for fully or partially surrounding the interior space. The housing may also provide a basis for attachment and/or holding one or more further components or elements. The housing may be formed by two or even more components of the medical device. The two or even more components may at least partially, preferably fully, surround the tight chamber.

As outlined above, the housing at least partially surrounds the at least one tight chamber. The term “chamber” as used herein is a broad term and is to be given its ordinary and customary meaning to a person of ordinary skill in the art and is not to be limited to a special or customized meaning. The term specifically may refer, without limitation, to an arbitrary partially or fully enclosed space that may be usable to contain and/or store objects. The housing may comprise one or even more than one chambers such as at least two chambers. The at least two chambers may be separate chambers. One, more than one or even all of the at least two chambers may be tight chambers, respectively. The chamber may be formed by a compartment of the housing. The term “compartment” as used herein is a broad term and is to be given its ordinary and customary meaning to a person of ordinary skill in the art and is not to be limited to a special or customized meaning. The term specifically may refer, without limitation, to an arbitrary subpart of a superior element creating a partially or fully enclosed space that may be usable to contain and/or store objects. The subpart may specifically be completely or at least to a large extent closed such that an interior of the compartment may be isolated from a surrounding environment. Exemplarily, the compartment may be separated from other parts of the superior element by one or more walls. Thus, within the housing, two or more compartments may be comprised which may fully or partially be separated from one another by one or more walls of the housing. Each compartment may comprise a continuous space or lumen configured for receiving one or more objects.

The term “tight chamber” as used herein is a broad term and is to be given its ordinary and customary meaning to a person of ordinary skill in the art and is not to be limited to a special or customized meaning. The term specifically may refer, without limitation, to a property of a chamber of being isolated from a surrounding environment such that a transfer of gas, fluids and/or solid elements is completely or at least to a large extent reduced. The tight chamber may also be referred to as sealed chamber. The tight chamber specifically may be a hermetically sealed chamber. The term “hermetically sealed chamber” as used herein is a broad term and is to be given its ordinary and customary meaning to a person of ordinary skill in the art and is not to be limited to a special or customized meaning. The term specifically may refer, without limitation, to an arbitrary chamber which is sealed or closed in a way such that a transfer of a gas or a fluid between an interior lumen of the chamber and a surrounding environment is prevented completely.

Specifically, the tight chamber may be a sterile chamber. The term “sterile” may generally refer to a property of an arbitrary object or space of being at least to a large extent free from all forms of life and/or other biological agents such as prions, viruses, fungi, bacteria or spore forms. Thus, the sterile object or space may be treated by at least one sterilization process that eliminates and/or deactivates the forms of life and/or the other biological agents. The sterilization process may comprise one or more of the following techniques: heating, chemical treatment, irradiation, high pressure filtration. However, other techniques are feasible.

The term “at least partially surround,” also referred to as “at least partially enclose,” as used herein is a broad term and is to be given its ordinary and customary meaning to a person of ordinary skill in the art and is not to be limited to a special or customized meaning. The term specifically may refer, without limitation, to embodiments wherein an object fully surrounds one or more further components and to embodiments wherein the object surrounds at least a part of the one or more further components. As outlined above, the housing of the medical device at least partially surrounds the tight chamber. In case the housing of the medical device fully surrounds the tight chamber, the tight chamber may be fully surrounded by at least one wall of the housing. However, in case the housing of the medical device partially surrounds the tight chamber, a tightness of the tight chamber may additionally be received by further components of the medical device.

As outlined above, the tight chamber is configured for sustaining a pressure difference between at least one interior lumen of the tight chamber and a surrounding environment. Due to a tightness of the tight chamber, a transfer of gas between the interior lumen of the tight chamber and the surrounding environment may be prevented at least to a large extent and the pressure difference between the interior lumen of the tight chamber and the surrounding environment may be sustained. To the contrary, in case of a leaky chamber, transfer of gas between the interior lumen of the chamber and the surrounding environment may occur and the pressure difference between the interior lumen of the tight chamber and the surrounding environment may not be sustained.

The invasive portion may be at least partially received in the tight chamber. Specifically, the tight chamber may be a hermetically sealed sterile chamber. Thus, the housing may be a housing for receiving the invasive portion of the medical device. The housing may at least partially be formed by at least one sterility cap. Further details on the sterility cap are given below in more detail.

The medical device may comprise at least one invasive portion compartment. The invasive portion of the medical device may be at least partially received in the invasive portion compartment. The medical device may comprise the at least one sterility cap, specifically at least one detachable sterility cap, at least partially surrounding at least a part of the invasive portion of the medical device. The sterility cap may at least partially surround the invasive portion compartment. The sterility cap may specifically at least partially surround the tight chamber. The tight chamber may be surrounded by the invasive portion compartment.

The term “cap” as used herein is a broad term and is to be given its ordinary and customary meaning to a person of ordinary skill in the art and is not to be limited to a special or customized meaning. The term specifically may refer, without limitation, to an arbitrary shaped element configured for fully or partially enclosing one or more objects and/or for providing protection for these one or more objects, such as against mechanical influence and/or humidity. The term “sterility cap” as used herein is a broad term and is to be given its ordinary and customary meaning to a person of ordinary skill in the art and is not to be limited to a special or customized meaning. The term specifically may refer, without limitation, to an element such as a cover which is configured for maintaining a sterile atmosphere in a space fully or partially surrounded by the element. The sterility cap, as an example, may be a rigid sterility cap, e.g., made of a rigid plastic material and/or a metal.

The sterility cap specifically may be essentially rotationally symmetric, e.g., by having an axial rotational symmetry about an axis such as a cylinder axis or axis of extension. The term “essentially rotationally symmetric” as used herein is a broad term and is to be given its ordinary and customary meaning to a person of ordinary skill in the art and is not to be limited to a special or customized meaning. The term specifically may refer, without limitation, to the fact that the sterility cap may be fully rotationally symmetric or may comprise at least one part being rotationally symmetric, whereas other parts of the sterility cap may exhibit a form diverging from the rotational symmetry. The sterility cap may be designed as a cylinder, a hemisphere or as a dome. The cap may have a shape that is adapted to a shape of the invasive portion of the medical device.

The sterility cap, as an example, may have an elongated shape, with a length exceeding its diameter or equivalent diameter by at least a factor of 2, more preferably by at least a factor of five. The sterility cap, as an example, may have a length of 5 to 20 mm, e.g., a length of 10 to 15 mm. Further, the sterility cap, as an example, may have a diameter of 2 mm to 8 mm, preferably of 4 mm to 7 mm and most preferably of 5 mm to 6 mm. Exemplarily, the sterility cap may have a length of 13 mm and a diameter of 5.5 mm. However, also other dimensions may be possible.

The term “detachable” may refer to a property of an element of being removable from an arbitrary object. Thereby, a close bonding or contact between the element and the object may be disconnected. Generally, the element may be removable in a reversible manner wherein the element may be attachable and detachable from the object or in an irreversible manner wherein the element may not be attachable to the object after detachment. By detachment of the sterility cap, the invasive portion of the medical device may be exposed and the medical device may be applied by the user or the patient.

As outlined above, the insertion component specifically may comprise the at least one insertion cannula. The insertion cannula may fully or partially be received in the sterility cap. The sterility cap, as an example, may have an elongated shape, with a closed end and an open end, with the insertion cannula protruding from the open end into the sterility cap, with the tip of the insertion cannula facing the closed end. The analyte sensor may partially be received in the insertion cannula, such as in a slot of the insertion cannula. The insertion component may further comprise at least one holder for the insertion cannula, wherein the holder, the insertion cannula and the sterility cap form components of a sterile container for the analyte sensor. The holder, as an example, may comprise a rigid component connected to a proximal end of the insertion cannula, i.e., to an end of the insertion cannula opposing the tip of the insertion cannula. The insertion cannula, as an example, may be connected to the holder by gluing and/or by injection molding and/or, e.g., by other means of material engagement. The holder, as an example, may have a cylindrical shape.

Specifically in case the medical device is the device for detecting the at least one analyte in the body fluid of the user, the invasive portion may comprise the analyte sensor and the insertion component. The medical device may further comprise the at least one holder for the insertion component. The holder for the insertion component and the sterility cap may at least partially surround the tight chamber. The housing of the medical device may be formed by the holder for the insertion component and the sterility cap. Thus, the holder for the insertion component and the sterility cap may form a sterile container for the analyte sensor and for the insertion component. However, also other components of the housing may contribute to surrounding and/or forming the tight chamber.

Specifically in case the medical device is the medication device for delivering the at least one therapeutical medical fluid to the user, the invasive portion may comprise the infusion cannula. The medical device may further comprise at least one holder for the infusion cannula. The tight chamber may be at least partially surrounded by the sterility cap and by the holder for the infusion cannula. The housing of the medical device may be formed by the holder for the infusion cannula and the sterility cap. Thus, the holder for the infusion cannula and the sterility cap may form a sterile container for the infusion cannula. However, also other components of the housing may contribute to surrounding and/or forming the tight chamber.

The medical device may further comprise at least one patch configured to be mounted onto a skin of the user. The patch specifically may comprise a plate which may be used as a support of other components of the medical device such as of the electronic components. Further, the patch may be configured for attaching the components of the medical device to the skin site of the user. For this purpose, the patch may comprise at least one adhesive surface and/or at least one adhesive strip or plaster. The patch may comprise at least one patch base. The holder of the insertion component or of the infusion cannula, the sterility cap and the patch base may at least partially surround the tight chamber. The housing of the medical device may be formed by the holder for the infusion cannula or for the insertion component, the sterility cap and the patch base. Also other embodiments may be feasible.

The medical device may further comprise at least one electronics unit. The electronics unit may comprise at least one electronics component. The electronics unit may further comprise at least one electronics unit housing. The electronics component may be received in the electronics unit housing. The medical device may comprise at least one electronics component compartment. The electronics component of the medical device may be at least partially received in the electronics component compartment. The tight chamber may be surrounded by the electronics unit housing. The housing may be the electronics unit housing.

The term “electronics unit” as used herein is a broad term and is to be given its ordinary and customary meaning to a person of ordinary skill in the art and is not to be limited to a special or customized meaning. The term specifically may refer, without limitation, to an arbitrary unit, such as a unit which may be handled as a single piece, which is configured for performing at least one electronic function. Specifically, in case the medical device is the medical device for detecting the analyte in the body fluid of the user, the electronics unit may have at least one interface for being connected to the analyte sensor, wherein the electronics unit may provide at least one electronic function interacting with the analyte sensor, such as at least one measurement function.

Alternatively to the embodiment with an electronics component compartment, the electronics unit may comprise or may be a plug-in connector or an electrical connection. Thus, the electronics unit may comprise an electrical and/or mechanical interface. Thus, there may be no further compartment besides the invasive portion compartment.

Specifically, in case the medical device is the medical device for detecting the analyte in the body fluid of the user, the electronics unit may be configured for one or more of determining and/or controlling a detection of the analyte and/or transmitting measurement data to another component. Specifically, the electronics component may be configured for one or more of performing a measurement with the analyte sensor, performing a voltage measurement, performing a current measurement, recording sensor signals, storing measurement signals and/or measurement data, transmitting sensor signals to another component. Thus, the electronics unit specifically may comprise at least one of: a voltmeter, an ammeter, a potentiostat, a voltage source, a current source, a signal receiver, a signal transmitter, an analog-digital converter, an electronic filter, a data storage device, an energy storage device.

The analyte sensor may be partially enclosed by the electronics unit housing. The electronics unit specifically may comprise the at least one electronics unit housing, wherein the analyte sensor, e.g., with a proximal end, may protrude into the electronics unit housing and may be electrically connected with at least one electronic component within the electronics unit housing. As an example, the proximal end and/or at least one contact portion of the analyte sensor may protrude into the electronics unit housing and, therein, may be electrically connected to the at least one electronic component, such as to at least one printed circuit board and/or at least one contact portion of the electronics unit, e.g., by one or more of a soldering connection, a bonding connection, a plug, a clamping connection or the like. The electronics unit specifically may be used as a transmitter for transmitting measurement data to at least one external device, such as to at least one receiver, e.g., wirelessly.

The electronics unit housing may be a sealed housing. The electronics unit housing commonly does not have to meet the requirements of sterile tightness, but commonly has to be waterproof and dustproof.

Specifically in case the medical device is the medication device for delivering the at least one therapeutical medical fluid to the user, the medical device may comprise the following components:
at least one medication reservoir configured for storing the at least one therapeutical medical fluid;
at least one infusion cannula such as described above or as will further be described below in more detail; and
at least one medication pump configured for transferring the therapeutical medical fluid from the medication reservoir to the infusion cannula.

Specifically in case the medical device is the device for detecting the at least one analyte in the body fluid of the device for detecting user, the medical device may comprise the following components:
at least one analyte sensor such as described above or as will further be described below in more detail;
at least one electronics unit such as described above or as will further be described below in more detail, wherein the electronics unit is electrically connected to the analyte sensor; and
at least one insertion component such as described above or as will further be described below in more detail, wherein the insertion component is configured for inserting the analyte sensor into the body tissue of the user.

Specifically, the medical device may comprise at least two of the housings. Specifically, the medical device may have at least one first housing having at least one first tight chamber. The sterility cap may at least partially surround the first tight chamber. Thus, the first housing may comprise the sterility cap. Further, the medical device may specifically have at least one second housing having at least one second tight chamber. The second housing may be the electronics unit housing and the electronics component may be received in the second tight chamber. The medical device may comprise at least one first deformable component and at least one second deformable component. A wall of the sterility cap may comprise the first deformable component. A wall of the electronics unit housing may comprise the second deformable component.

As outlined above, the medical device may comprise the invasive portion compartment and the electronics component compartment. The invasive portion compartment and the electronics component compartment may be two separate compartments. An interior lumen of the invasive portion compartment may be isolated from an interior lumen of the electronics component compartment. The invasive portion compartment and the electronics component compartment may be separated from one another by one or more walls of the medical device. Yet, optionally, the invasive portion compartment and the electronics component compartment may share at least one common wall. The invasive portion may be received at least partially in the invasive portion compartment. However, a part of the invasive portion may be received in the electronics component compartment. The invasive portion compartment may comprise at least one sealed opening, such as for leading a portion of the invasive portion such as the analyte sensor out of the invasive portion compartment, in order to be operably connected to the at least one electronic component received in the electronics component compartment.

The term “deformable component,” as used herein, is a broad term and is to be given its ordinary and customary meaning to a person of ordinary skill in the art and is not to be limited to a special or customized meaning. The term specifically may refer, without limitation, to an arbitrary component which may change its shape as a result of an action of an external force or mechanical stress. The deformation specifically may be described as a change in length, i.e., as an elongation. The deformation may refer to a reversible elastic deformation or to an irreversible plastic deformation. A reversible elastic deformable material may change its shape when a force is applied and may return to its original shape when the applied force is removed. An irreversible plastic deformation may take place in case an elastic limit of a material is reached. A prerequisite for this is that the material is formable and can absorb deformation energy. An associated property of such a material is also called ductility. Specifically, the deformable component may be deformable under pressure.

Monitoring the integrity of the housing may be performed by applying a negative or a positive pressure. The negative pressure may be naturally limited to a pressure difference of max. approx. 100000 Pa. Exemplarily, the monitoring of an integrity of the housing may be performed at a differential pressure which is larger than 80000 Pa (i.e., 20000 Pa abs).

The monitoring of the integrity of the housing may be performed by applying the positive pressure. Thus, principally, any pressure may be applied. However, the applied pressure may be based on a service pressure of the medical device and its mechanical properties. Exemplarily, the monitoring of an integrity of the housing may be performed at a differential pressure of 100000 Pa to 400000 Pa (i.e., 500000 Pa abs). At a relative test pressure of 400000 Pa a deformation of a medical device without the deformable component in the range of 10 nm to 100 nm may be expected. Such a deformation may be too small to perform a test with usual measuring methods in the production environment.

The deformable component may have a flat shape. The deformable component may have a large areal extent in relation to a thickness of a deformable component. Exemplarily, the deformable component may have a length and/or a width which exceed(s) the thickness of the deformable component by at least a factor of 2, preferably by at least a factor of 10 and most preferably by at least a factor of 20. However, also other dimensions may be feasible.

The deformable component may exemplarily have a thickness of 0.05 mm to 2.0 mm, preferably of 0.1 mm to 1.0 mm and most preferably of 0.3 mm. Further, the deformable component may have a length of 1 mm to 5 mm, preferably of 2 mm to 4 mm and most preferably of 3 mm. Further, the deformable component may have a width of 1 mm to 5 mm, preferably of 2 mm to 4 mm and most preferably of 3 mm. Exemplarily, the deformable component may have a thickness of 0.3 mm, a length of 3 mm and a width of 3 mm. However, also other dimensions may be possible.

The sterility cap, specifically the wall of the sterility cap, may exemplarily have a thickness of 0.2 mm to 5 mm, preferably of 0.5 mm to 3 mm. Specifically, the wall of the sterility cap may have a thickness of 0.5 mm to 3 mm and the deformable component may have a thickness of 0.1 mm to 1.0 mm. However, also other dimensions may be possible.

The electronics unit housing, specifically the wall of the electronics unit housing, may exemplarily have a thickness of 0.08 mm to 5 mm, preferably of 0.1 mm to 3 mm. Specifically, the wall of the sterility cap may have a thickness of 0.1 mm to 3 mm and the deformable component may have a thickness of 0.1 mm to 1.0 mm. However, also other dimensions may be possible.

The deformable component may have an arbitrary shape. Exemplarily, the deformable component may have a round basic shape such as a circular basic shape, an ellipsoidal basic shape, an oval shape, a rectangular basic shape, specifically a rectangular basic shape with rounded corners. However, also other embodiments may be feasible.

The deformable component may exemplarily be made of at least one material selected from the group consisting of: acrylonitrile butadiene styrene (ABS), polypropylene, polystyrene, polyamide, polycarbonate, polyethylene; polymethyl methacrylate (PMMA), polyoxymethylene (POM), thermoplastic elastomers (TPE), silicone. However, also other materials would be feasible.

Exemplarily, the deformable component may comprise at least one material having a mtensile strength of: 40 MPa to 180 MPa.

The housing, specifically the electronics unit housing and/or the sterility cap, may exemplarily be at least partially made of polycarbonate. However, also other materials may be possible.

The deformable component may be a predefined material strength and/or a predefined thickness. The term “predefined” specifically refers to the circumstance that the material strength and/or the thickness are defined prior to monitoring an integrity of the housing of the medical device. Thus, the material strength and/or the thickness are predetermined and known prior to monitoring the integrity of the housing of the medical device. Exemplarily, the material strength may be adjusted such as through a choice of material and/or the thickness may be adjusted such as through a choice of a design of the deformable component. The term “material strength” may generally refer to a material property which describes a mechanical resistance that a material offers to plastic deformation or separation. The material strength may be determined from a stress-strain diagram. Depending on a choice of material, a material condition, a temperature, a load and a loading speed, different strengths may be achieved.

At least one wall of the housing, specifically an outer wall of the housing, may comprise the deformable component. The housing, specifically the wall of the housing, specifically the outer wall of the housing, may comprise at least one recess such as at least one cut-out. The deformable component may be located or received in the recess or the cut-out. The recess may also be referred to as window of the housing, specifically of the wall of the housing. The deformable component may form a part of the wall of the housing. A remaining part of the wall of the housing may at least partially, preferably fully, surround the deformable component.

The housing and the deformable component may be designed integrally. The term “integrally” may refer to a state wherein two or more components are arranged in a space-saving or compact manner. At least one of the two or more components may be permanently built into at least another one of the two or more components. Further, the two or more components may be designed in a complementary manner such that the components may be able to interact with each other. Exemplarily, a wall of the housing and the deformable component may form a single piece.

Specifically, the housing and the deformable component may be manufactured in one piece. In this case, the deformable element may be distinguishable only by its offset, function-oriented contour. Specifically, the housing and the deformable component may be manufactured via injection molding. The housing and the deformable component may be characterized by their special geometry. For example, the housing may have a wall which is thicker than a wall of the deformable component.

A manufacturing of the housing and the deformable component in one piece but from different materials conducted by using two-component injection molding. Thereby, each of the housing and the deformable component may be made from a material adapted to its specific function. For example, the housing may be made from at least one solid material and the deformable component may be made from at least one elastic material. A sealing between the housing and the deformable component may be achieved by melting the two materials during the injection molding.

The deformable component may be sealed against the remaining wall of the housing. Thus, the tight chamber may be formed. Exemplarily, there may be adhesive bonds between the housing and the deformable component such as via curing adhesives. Further, also ultrasonic or laser welded joints may be applied. Also other methods may be possible.

Specifically, as outlined above, the invasive portion may be at least partially received in the tight chamber and the housing may at least partially be formed by the least one sterility cap. The sterility cap may comprise the deformable component. The deformable component may form part of a wall, specifically of an outer wall, of the sterility cap. Specifically, the deformable component may form part of a wall, specifically of an outer wall, of the sterility cap surrounding a rotational axis of the sterility cap.

Further, additionally or alternatively, the housing may be the electronics unit housing and the electronics component may be received in the tight chamber. The deformable component may form part of a wall, specifically of an outer wall, of the electronics unit housing.

As outlined above, the housing is configured such that a state of deformation of the deformable component provides for a measurable indication of the pressure difference between the interior lumen of the tight chamber and the surrounding environment. In case the medical device is placed into a testing chamber and a test pressure may be applied to a testing chamber, the deformable component may change its shape as a result of the applied test pressure. The test pressure may be larger or smaller than the ambient pressure. In dependence of the test pressure and in case of a tight chamber, the deformable component may turn inward the interior lumen of the tight chamber or may turn outward, i.e., towards the surrounding environment. The turning inward the interior lumen of the tight chamber and the turning outward of the deformable component may be referred to as a deformation of the deformable component. The state of deformation may change over time. Thus, the state of deformation may be detected over time for a purpose of evaluating the integrity of the housing of the medical device. Thus, the deformation of the deformable component may be dependent on the pressure difference between the interior lumen of the tight chamber and the surrounding environment. Thus, when detecting the state of deformation of the deformable component it may be associated with the pressure difference between the interior lumen of the tight chamber and the surrounding environment. In dependence of the test pressure and in case of a leaky chamber, the deformable component may not deform due to a pressure compensation between the interior lumen and the surrounding environment.

The medical device may further comprise at least one integrated deformation sensor configured for measuring the state of the deformation of the deformable component. The term “sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one condition or for measuring at least one measurement variable. The term “deformation sensor” as used herein is a broad term and is to be given its ordinary and customary meaning to a person of ordinary skill in the art and is not to be limited to a special or customized meaning. The term specifically may refer, without limitation, to an arbitrary element or device configured for detecting at least one deformation or displacement of an arbitrary object. Specifically, the deformation sensor may be configured for detecting a bulge of the object. The term “integrated sensor” as used herein is a broad term and is to be given its ordinary and customary meaning to a person of ordinary skill in the art and is not to be limited to a special or customized meaning. The term specifically may refer, without limitation, to an arbitrary sensor with is configured to be, preferably fixedly, attached to or to be mounted on an arbitrary test specimen. Thus, the integrated deformation sensor may also be referred to as on-board deformation sensor. Specifically, the integrated deformation sensor may be attached to a surface of the deformable component sensor and/or to a surface of the housing, specifically to a surface of the sterility cap or to a surface of the electronics unit housing. Specifically, the integrated deformation sensor may fully or partially cover the surface of the deformable component. The surface of the deformable component may specifically be an outer surface facing the outer environment.

The integrated deformation sensor may comprise at least one extension-sensitive color layer. The extension-sensitive color layer may be configured for changing color in dependence of the deformation of the deformable component. A first color may indicate an intact tightness of the tight chamber. A second color which may be different from the first color may indicate a defective tightness of the tight chamber. A color change of the extension-sensitive color layer may be visible by the user or by the inspector by eye. Thus, an application of the at least one extension-sensitive color layer may be an embodiment without electronic evaluation of the tightness of the tight chamber.

Further, additionally or alternative, the integrated deformation sensor may be selected from the group consisting of: a strain gauge; a switching contact; an optical distance measurement sensor such a reflection measurement sensor or an angle measurement sensor; a capacitive sensor. However, also other methods may be possible such as a triangulation method or a resistance measurement of a thin film.

As a special embodiment, the deformable component may be or may comprise a foil with special optical properties. With a suitable foil, a low-cost optical sensor may detect a curvature of the foil with aid of an angle of reflection.

The integrated deformation sensor may comprise at least one transmitter for transmitting measurement values to at least one external receiver.

Specifically, the medical device may have at least one movable component. The moveable component may be a cap closing the tight chamber of the housing. The medical device, specifically the movable component itself, may comprise at least one sealing element. The sealing element may be made of at least one elastomeric material. The sealing element may be configured for sealing the movable component against the tight chamber. The movable component may be detachably connected to the housing. The movable component and the housing may form a multi-part design. The movable component may be detachably inserted and joined to the housing with the aid of the sealing element. Elastic properties of the sealing element may allow the movable component to move in relation to the housing. Due to an applied relative pressure difference, the movable component may experience a force corresponding to the pressure difference and may move within an elastic displacement of the sealing element.

Exemplarily, the moveable component may be a cap closing the invasive portion compartment. Exemplarily, the sterility cap may form a detachable lower cap of the invasive portion compartment and the movable component may form a detachable upper cap of the invasive portion compartment. The electronics unit housing may form an intermediate component of the medical device being disposed in between the sterility cap and the moveable component. The sterility cap and the moveable component may be located on opposing sides of the electronics unit housing. The sealing element may be arranged in the electronics unit housing. Alternatively, the sealing element may be part of the moveable component. The sealing element may be configured for sealing the movable component against the electronics unit housing. The movable component may be detachably connected to the electronics unit housing. The movable component and the electronics unit housing may form a multi-part design. The movable component may be detachably inserted and joined to the electronics unit housing with the aid of the sealing element. Elastic properties of the sealing element may allow the movable component to move in relation to the electronics unit housing. Due to an applied relative pressure difference, the movable component may experience a force corresponding to the pressure difference and may move within an elastic displacement of the sealing element.

In a further aspect of this disclosure, a kit is disclosed.

The term “kit” as used herein is a broad term and is to be given its ordinary and customary meaning to a person of ordinary skill in the art and is not to be limited to a special or customized meaning. The term specifically may refer, without limitation, to a group of at least two elements which may interact with each other in order to fulfill at least one common function. The at least two components may be handled independently or may be coupled, connectable or integrable in order to form a common device.

The kit comprises the medical device as described above or as will further be described below in more detail. Thus, for possible definitions and options, reference may be made to the disclosure of the medical device according to this disclosure.

Further, the kit comprises at least one external deformation sensor configured for measuring the state of the deformation of the deformable component. The term “external sensor” as used herein is a broad term and is to be given its ordinary and customary meaning to a person of ordinary skill in the art and is not to be limited to a special or customized meaning. The term specifically may refer, without limitation, to an arbitrary sensor which is configured to be provided separately from a test specimen. Specifically, the external sensor may be arranged in a distance to the test specimen. The external deformation sensor may specifically be a displacement sensor. The displacement sensor may be configured for measuring a distance between an object and a reference point or a change in a length. The displacement sensor may exemplarily be selected from the group consisting of: an inductive sensor; an incremental encoder; a laser rangefinder. However, also other embodiments may be feasible.

The medical kit may further comprise at least one integrity evaluation unit configured for deriving at least one item of integrity information from the state of deformation. The term “evaluation unit” as used herein is a broad term and is to be given its ordinary and customary meaning to a person of ordinary skill in the art and is not to be limited to a special or customized meaning. The term specifically may refer, without limitation, to an arbitrary functional element configured for analyzing and/or processing data. The evaluation unit may specifically analyze and/or process measurement data, e.g., the measurement results as generated by the impedance measurement unit. The evaluation unit may in particular comprise at least one processor. The processor may specifically be configured, such as by software programming, for performing one or more evaluation operations on the measurement results.

The term “integrity” as used herein is a broad term and is to be given its ordinary and customary meaning to a person of ordinary skill in the art and is not to be limited to a special or customized meaning. The term specifically may refer, without limitation, to a property of an object of fulfilling one or more integrity criteria. Specifically, the integrity criteria may refer to predefined properties of the object. More specifically, the integrity criteria may refer to a leakage tightness of the object. The object may specifically have at least one lumen which is surrounded by one or more walls.

The kit may further comprise at least one testing chamber. The term “testing chamber” as used herein is a broad term and is to be given its ordinary and customary meaning to a person of ordinary skill in the art and is not to be limited to a special or customized meaning. The term specifically may refer, without limitation, to an arbitrary partially or fully enclosed space that may be pressurized with a defined pressure. The defined pressure may also be referred to as test pressure. The test pressure may be larger or smaller than the ambient pressure. The testing chamber may be a tight chamber. The testing chamber and the tight chamber surrounded by the housing of the medical device may be separate chambers. The medical device may be fully receivable within the testing chamber. The testing chamber may not be part of the medical device.

In a further aspect of this disclosure, a method for monitoring an integrity of a housing of a medical device is disclosed.

The term “monitoring” as used herein is a broad term and is to be given its ordinary and customary meaning to a person of ordinary skill in the art and is not to be limited to a special or customized meaning. The term specifically may refer, without limitation, to one or more of detecting at least one property of an object and detecting a change of the at least one property of the object over time. As an example, the at least one property may be a physical property. The term “method for monitoring an integrity of a housing” as used herein is a broad term and is to be given its ordinary and customary meaning to a person of ordinary skill in the art and is not to be limited to a special or customized meaning. The term specifically may refer, without limitation, to an arbitrary process wherein at least one property of a housing of fulfilling one or more integrity criteria is monitored. Thus, the term may specifically refer to monitoring at least one predefined property of the housing and more specifically to a monitoring of a leakage tightness of the housing. A structural soundness and performance of the housing may be monitored. The monitoring of the integrity may specifically be conducted at a manufacturer's end. The monitoring of the integrity may specifically be performed by the manufacturer during product development and/or part of manufacturing quality controls. It may be ensured that the housing may function according to manufacturer's sterility claims and that there are no leaks or defects in the housing.

The method comprises the steps disclosed in the following. The steps specifically may be performed in the given order. Still, a different order is possible. The method may comprise additional steps which are not mentioned. It is further possible to perform one or more of the method steps repeatedly. Further, two or more of the method steps may be performed in a timely overlapping fashion or simultaneously.

The method comprises the following steps:
a) providing a medical device as described above or as will further be described below in more detail;
b) placing the medical device into a testing chamber and applying a test pressure to the testing chamber;
c) detecting the state of deformation of the deformable component, preferably over time; and
d) evaluating the integrity of the housing of the medical device based on the state of deformation of the deformable component.

Thus, for possible definitions and options of the medical device reference may be made to the disclosure of the medical device according to this disclosure.

The test pressure may be larger or smaller than the ambient pressure.

The testing chamber may comprise at least one external deformation sensor configured for measuring the state of deformation of the deformable component. For further details on the external deformation sensor, reference is made to the description above. Before step c) is conducted, the external deformation sensor may be tared at normal pressure.

Additionally or alternatively, the medical device may further comprise at least one integrated deformation sensor. For further details on the integrated deformation sensor, reference is made to the description above. In step c) the integrated deformation sensor may detect the state of the deformation of the deformable component.

In the case of a tight chamber, the deformable component may undergo a deformation. In the case of a faulty tight chamber, thus in case of a leaky chamber, a same pressure occurs inside the chamber due to a leak. Due to the pressure compensation, no deformation takes place on the deformable component. If a deflection of the deformable component is recorded over time, i.e., a dynamic measurement, it is possible to draw conclusions about a size of the leakage from a shape of a deflection over time result curve showing a deflection.

In addition, the deformable component may return to an initial position when the test pressure is released. The extent to which the deformable component returns to its original position may allow conclusions to be drawn about the tightness of the deformable component. This may be used as a further evaluation criterion. In contrast to a pure consideration of the displacement, undesirable measurement and artifacts resulting from the deformable component may be easily compensated mathematically by forming a ratio of the displacement movement and a remaining reset position. This means that a relative method can be used to evaluate an absolute measurement.

Further, the medical device may have the at least one movable component. For further details, reference may be made to the description above. The method may further comprise detecting movement of the movable element in relation to its reset position and evaluating the integrity of the housing.

Further disclosed and proposed herein is a computer program including computer-executable instructions for performing the method, specifically one or both of steps c) and d), according to this disclosure in one or more of the embodiments enclosed herein when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps c) and d) as indicated above may be performed by using a computer or a computer network, preferably by using a computer program.

Further disclosed and proposed herein is a computer program product having program code means, in order to perform the method, specifically one or even all of method steps c) and d), according to this disclosure in one or more of the embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the method, specifically one or even all of method steps c) and d), according to one or more of the embodiments disclosed herein.

Further disclosed and proposed herein is a non-transient computer-readable medium including instructions that, when executed by one or more processors, cause the one or more processors to perform the method, specifically one or even all of method steps c) and d), according to one or more of the embodiments disclosed herein.

Further disclosed and proposed herein is a computer program product with program code means stored on a machine-readable carrier, in order to perform the method, specifically one or even all of method steps c) and d), according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the method, specifically one or even all of method steps c) and d), according to one or more of the embodiments disclosed herein.

Referring to the computer-implemented aspects of this disclosure, one or more of the method steps, specifically one or even all of method steps c) and d), of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the method, specifically one or even all of method steps c) and d), according to one of the embodiments described in this description,
a computer loadable data structure that is adapted to perform the method, specifically one or even all of method steps c) and d), according to one of the embodiments described in this description while the data structure is being executed on a computer,
a computer program, wherein the computer program is adapted to perform the method, specifically one or even all of method steps c) and d), according to one of the embodiments described in this description while the program is being executed on a computer,
a computer program comprising program means for performing the method, specifically one or even all of method steps c) and d), according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method, specifically one or even all of method steps c) and d), according to one of the embodiments described in this description after having been loaded into a main and/or working storage of a computer or of a computer network, and
a computer program product having program code means, wherein the program code means can be stored or are stored on a storage medium, for performing the method, specifically one or even all of method steps c) and d), according to one of the embodiments described in this description, if the program code means are executed on a computer or on a computer network.

The methods and devices according to this disclosure provide a large number of advantages over known methods and devices. Commonly, tightness measurements of a device are used as an indirect measurement for testing device integrity. However, for complex devices comprising different chambers, such as continuous glucose monitoring devices, a specific measurement is difficult via such methods. This disclosure offers a method and medical device for a device specific integrity measurement via wall deformation under pressure.

Presently, the inventive device with a specific wall configuration such as a membrane-like functional area enables an integrity detection via a pressure chamber over time. Consequently, this leads to a selective measurement of the integrity, e.g., sterility/tightness of medical devices. The method can, for example, be applied in continuous glucose monitoring systems (CGM devices) or insulin delivery systems (IDS, insulin pump).

A selective measurement of the integrity, e.g., sterility/tightness, of medical devices may be possible. Furthermore, a 100 percent control of the devices (as in-process or release control) may be enabled. Release and in-process control tests may be simplified since an indicator may be mounted on the medial device.

An indirect proof may be provided by an integrity of the housing. The analyte sensor may be placed in the sterility cap which may be part of the product, e.g., the medical device. This means that part of the housing of the medical device may form a hermetically sealed sterile chamber in which the analyte sensor is located. An adapted design of the medical device may be provided, which enables a site-selective leak test. For this purpose, the tight chamber may be designed in such a way that it has a deformable outer wall at at least one point. Each further volume of the medical device that is to be tested for leak tightness may also be equipped with a deformable outer wall. To test the tightness of the chamber(s), the medical device may be placed in a closed testing chamber to which the test pressure is applied. This test pressure can be selected larger or smaller than the ambient pressure. The deformable outer wall of the medical device may be scanned with a displacement measurement sensor. In the case of a tight chamber, the deformable outer wall may undergo a deformation. In the case of a leaky medical device, the same pressure occurs inside the chamber to be tested (in this case either a chamber formed by the sterility cap or a chamber formed by the electronics unit housing) as in the volume of the testing chamber due to the leak. Due to the pressure compensation, no deformation takes place on the deformable wall. If a wall deflection is recorded over time, i.e., a dynamic measurement, it is possible to draw conclusions about the size of the leakage from a shape of a “deflection over time” result curve. By this type of design of the medical device, the medical device itself may be used as a sensor for its own leakage. A “normal” body surface may be elevated to a status of a functional surface for testing. Thus, a defined material strength, wall thickness, etc. may be specified by the design, resulting in a defined measurement effect. This kind of testing can be done within a very short measuring time, which is advantageous for in-process control.

It is also possible to have this deflection measured by the medical device itself. For example, strain gauges or other suitable sensor technology (switching contact) can be inserted into the medical device itself. During a test, the medical device may determine the deflection of the functional area with its own electronics and may pass a result wirelessly to a test system or to the user. In this way, for example, a statement about a sterile condition of the medical device is received.

Methods without electronics may also be conceivable as “on-board” sensor systems. If the functional surface is coated with a strain-sensitive, irreversible layer of paint, the color of the layer may indicate to an inspector whether the functional surface has already been deflected. An indicator color “red” or “green” then may signal an intact or defective sterile protection.

Summarizing and without excluding further possible embodiments, the following embodiments may be envisaged:

Embodiment 1: A medical device having at least one invasive portion, the medical device comprising at least one housing at least partially surrounding at least one tight chamber, the tight chamber being configured for sustaining a pressure difference between at least one interior lumen of the tight chamber and a surrounding environment, wherein the housing comprises at least one deformable component, wherein the housing is configured such that a state of deformation of the deformable component provides for a measurable indication of the pressure difference between the interior lumen of the tight chamber and the surrounding environment.

Embodiment 2: The medical device according to the preceding embodiment, wherein the medical device further comprises at least one integrated deformation sensor configured for measuring the state of the deformation of the deformable component.

Embodiment 3: The medical device according to the preceding embodiment, wherein the integrated deformation sensor comprises at least one extension-sensitive color layer, wherein the extension-sensitive color layer is configured for changing a color in dependence of the deformation of the deformable component.

Embodiment 4: The medical device according to any one of the two preceding embodiments, wherein the integrated deformation sensor is selected from the group consisting of: a strain gauge; a switching contact; an optical distance measurement sensor such a reflection measurement sensor or an angle measurement sensor; a capacitive sensor.

Embodiment 5: The medical device according to any one of the three preceding embodiments, wherein the integrated deformation sensor comprises at least one transmitter for transmitting measurement values to at least one external receiver.

Embodiment 6: The medical device according to any one of the four preceding embodiments, wherein the integrated deformation sensor is attached to a surface of the deformable component sensor and/or to a surface of the housing.

Embodiment 7: The medical device according to the preceding embodiment, wherein the integrated deformation sensor fully or partially covers the surface of the deformable component.

Embodiment 8: The medical device according to any one of the preceding embodiments, wherein a wall of the housing comprises the deformable component.

Embodiment 9: The medical device according to the embodiment, wherein the wall of the housing comprises at least one recess, wherein the deformable component is received in the recess.

Embodiment 10: The medical device according to any one of the preceding embodiments, wherein the deformable component forms part of the housing at least partially surrounding the tight chamber.

Embodiment 11: The medical device according to any one of the preceding embodiments, wherein the deformable component has a predefined material strength and/or a predefined thickness.

Embodiment 12: The medical device according to any one of the preceding embodiments, wherein the deformable component comprises at least one material having a tensile strength of: 40 MPa to 180 MPa.

Embodiment 13: The medical device according to any one of the preceding embodiments, wherein deformable component has a thickness of 0.1 mm to 1.0 mm.

Embodiment 14: The medical device according to any one of the preceding embodiments, wherein the deformable component is made of at least one material selected from the group consisting of: acrylonitrile butadiene styrene (ABS), polypropylene, polystyrene, polyamide, polycarbonate, polyethylene; polymethyl methacrylate (PMMA), polyoxymethylene (POM), thermoplastic elastomers (TPE), silicone.

Embodiment 15: The medical device according to any one of the preceding embodiments, wherein the housing is at least partially made of polycarbonate.

Embodiment 16: The medical device according to any one of the preceding embodiments, wherein the housing and the deformable component are designed integrally.

Embodiment 17: The medical device according to any one of the preceding embodiments, wherein the invasive portion is selected from the group consisting of: at least one analyte sensor for detecting at least one analyte in a body fluid of a user; at least one insertion component; at least one infusion cannula; at least one stimulating electrode.

Embodiment 18: The medical device according to the preceding embodiment, wherein the insertion component is selected from the group consisting of: an insertion needle; an insertion cannula.

Embodiment 19: The medical device according to any one of the preceding embodiments, wherein the invasive portion is at least partially received in the tight chamber.

Embodiment 20: The medical device according to any one of the preceding embodiments, wherein the medical device comprises at least one sterility cap at least partially surrounding the invasive portion.

Embodiment 21: The medical device according to the preceding embodiment, wherein the medical device further comprises at least one holder for the insertion component, wherein the holder and the sterility cap at least partially surround the tight chamber.

Embodiment 22: The medical device according to the preceding embodiment, wherein the medical device further comprises at least one patch configured to be mounted onto a skin of the user, wherein the patch comprises at least one patch base, wherein the holder, the sterility cap and the patch base at least partially surround the tight chamber.

Embodiment 23: The medical device according to any one of the three preceding embodiments, wherein the deformable component forms part of a wall of the sterility cap.

Embodiment 24: The medical device according to any one of the preceding embodiments, wherein the medical device further comprises at least one electronics unit, wherein the electronics unit comprises at least one electronics component, wherein the electronics unit further comprises at least one electronics unit housing, wherein the electronics component is received in the electronics unit housing.

Embodiment 25: The medical device according to the preceding embodiment, wherein the electronics unit housing is a sealed housing.

Embodiment 26: The medical device according to any one of the two preceding embodiments, wherein the housing is the electronics unit housing, wherein the electronics component is received in the tight chamber.

Embodiment 27: The medical device according to any one of the three preceding embodiments, wherein the deformable component forms part of a wall of the electronics unit housing.

Embodiment 28: The medical device according to any one of the preceding embodiments, wherein the medical device is selected from the group consisting of: a medication device for delivering at least one therapeutical medical fluid to a user, specifically a device for delivering insulin to the user; a device for detecting at least one analyte in a body fluid of the user, specifically a device for detecting glucose in the body fluid of the user.

Embodiment 29: A kit comprising the medical device according to any one of the preceding embodiments and at least one external deformation sensor configured for measuring the state of the deformation of the deformable component.

Embodiment 30: The kit according to the preceding embodiment, wherein the kit further comprises at least one integrity evaluation unit configured for deriving at least one item of integrity information from the state of deformation.

Embodiment 31: The kit according to any one of the two preceding embodiments, wherein the external deformation sensor is a displacement sensor, wherein the displacement sensor is selected from the group consisting of: an inductive sensor; an incremental encoder; a laser rangefinder.

Embodiment 32: A method for monitoring an integrity of a housing of a medical device, the method comprising:
a) providing a medical device according to any one of the preceding embodiments referring to a medical device;
b) placing the medical device into a testing chamber and applying a test pressure to the testing chamber;
c) detecting the state of deformation of the deformable component, preferably over time; and
d) evaluating the integrity of the housing of the medical device based on the state of deformation of the deformable component.

Embodiment 33: The method according to the preceding embodiment, wherein the test pressure is larger or smaller than the ambient pressure.

Embodiment 34: The method according to any one of the two preceding embodiments, wherein the testing chamber comprises at least one external deformation sensor configured for measuring the state of deformation of the deformable component.

Embodiment 35: The method according to the preceding embodiment, wherein before step c) the external deformation sensor is tared at normal pressure.

Embodiment 36: The method according to any one of the four preceding embodiments, the medical device further comprises at least one integrated deformation sensor, wherein in step c) the integrated deformation sensor detects the state of the deformation of the deformable component.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIGS. 1A and 1B show an exemplarily embodiment of a kit according to this disclosure in a cross-sectional view (FIG. 1A) and exemplary measurement results (FIG. 1B).

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

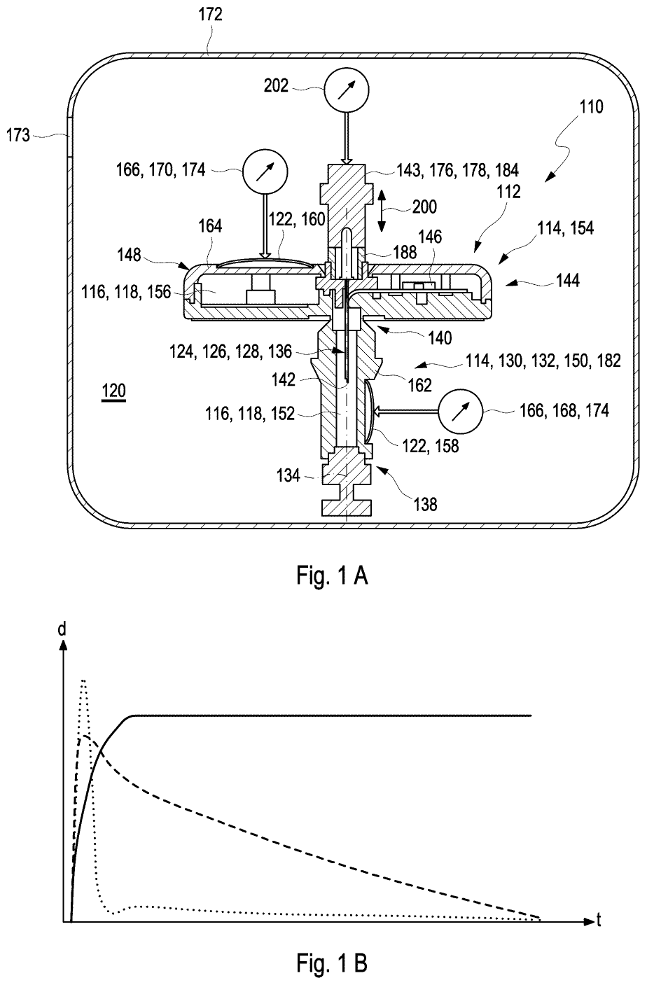

FIG. 1A shows an exemplarily embodiment of a kit 110 according to this disclosure in a cross-sectional view.

The kit 110 comprises at least one medical device 112. The medical device 112 comprises at least one housing 114 at least partially surrounding at least one tight chamber 116. The tight chamber 116 is configured for sustaining a pressure difference between at least one interior lumen 118 of the tight chamber and a surrounding environment 120. The housing 114 comprises at least one deformable component 122. The housing 14 is configured such that a state of deformation of the deformable component 122 provides for a measurable indication of the pressure difference between the interior lumen 118 of the tight chamber 116 and the surrounding environment 120. Further, the medical device comprises at least one invasive portion 124.

In the embodiment according to FIG. 1A, the medical device 112 is a device for detecting at least one analyte in a body fluid of a user, specifically a device for detecting glucose in the body fluid of the user. The invasive portion 124 may be an analyte sensor 126 for detecting at least one analyte in a body fluid of the user. Further, the invasive portion 124 may comprise at least one insertion component 128 for inserting the analyte sensor 126 into the body tissue of the user.

The medical device 112 may comprise at least one sterility cap 130, specifically at least one detachable sterility cap 132, at least partially surrounding at least a part of the invasive portion 124 of the medical device 112. The sterility cap 130, as an example, may have an elongated shape. The sterility cap 130 specifically may be essentially rotationally symmetric, e.g., by having an axial rotational symmetry about an axis 134 such as a cylinder axis or axis of extension.

The insertion component 128 specifically may comprise at least one insertion cannula 136. The insertion cannula 136 may fully or partially be received in the sterility cap 130. The sterility cap 130, as an example, may have a closed end 138 and an open end 140, with the insertion cannula 136 protruding from the open end 140 into the sterility cap 130, with a tip 142 of the insertion cannula 36 facing the closed end 138. The analyte sensor 126 may partially be received in the insertion cannula, such as in a slot of the insertion cannula 136. The insertion component 128 may further comprise at least one holder 143 for the insertion cannula 136, wherein the holder 142, the insertion cannula 136 and the sterility cap 130 form components of a sterile container for the analyte sensor 126.

The medical device 112 may further comprise at least one electronics unit 144. The electronics unit 144 may comprise at least one electronics component 146. The electronics unit 144 may further comprise at least one electronics unit housing 148. The electronics component 146 may be received in the electronics unit housing 148. The analyte sensor 126 may be partially enclosed by the electronics unit housing 148. The electronics unit 144 specifically may comprise the at least one electronics unit housing 148, wherein the analyte sensor 126, e.g., with a proximal end, may protrude into the electronics unit housing 148 and may be electrically connected with the at least one electronics component 146 within the electronics unit housing 148.

In the embodiment according to FIG. 1A, the medical device 112 may specifically comprise at least two of the housings 114 respectively having the at least one tight chamber 116. The medical device 112 may specifically have at least one first housing 150 having at least one first tight chamber 152. The sterility cap 130 may specifically at least partially surround the first tight chamber 152. Further, the medical device 112 may specifically have at least one second housing 150 having at least one second tight chamber 152. The second housing 154 may be the electronics unit housing 148 and the electronics component 146 may be received in the second tight chamber 156.

The medical device 112 may comprise at least one first deformable component 158 and at least one second deformable component 160. A wall 162 of the sterility cap 130 may comprise the first deformable component 158. A wall 164 of the electronics unit housing 148 may comprise the second deformable component 160.

Further, the kit 110 comprises at least one external deformation sensor 166 configured for measuring the state of the deformation of the deformable component 122. In the embodiment according to FIG. 1A, the kit 110 may comprise at least one first external deformation sensor 168 configured for measuring the state of the deformation of the first deformable component 158 and at least one second external deformation sensor 170 configured for measuring the state of the deformation of the second deformable component 160.

Further, the medical device 112 may have at least one movable component 176. Exemplarily, the moveable component 176 may be a cap 178 closing an invasive portion compartment. The cap 178 may correspond to the holder 143 of the insertion cannula 136. Exemplarily, the sterility cap 130 may form a detachable lower cap 182 of the invasive portion compartment and the movable component 176 may form a detachable upper cap 184 of the invasive portion compartment. The electronics unit housing 148 may form an intermediate component of the medical device 112 being disposed in between the sterility cap 130 and the moveable component 176. The sterility cap 130 and the moveable component 176 may be located on opposing sides of the electronics unit housing 148. A sealing element 188 may be arranged on the moveable component 176 and may be configured for sealing the movable component 176 against the electronics unit housing 148. The sealing element 188 may be arranged between the movable component 176 and the electronics unit housing 148. The movable component 176 may be detachably connected to the electronics unit housing 148. The movable component 176 and the electronics unit housing 148 may form a multi-part design. The movable component 176 may be detachably inserted and joined to the electronics unit housing 148 with the aid of the sealing element 188. Elastic properties of the sealing element 188 may allow the movable component 176 to move in relation to the electronics unit housing 148. Due to an applied relative pressure difference, the movable component 176 may experience a force corresponding to the pressure difference and may move within an elastic displacement of the sealing element 188 such as indicated via arrow 200 in FIG. 1A. A movement of the movable element 176 in relation to its reset position may be detected such as indicated in FIG. 1A with sensor 202 and the integrity of the housing may be evaluated.

The medical device 112 may be inserted into a testing chamber 172. A pressure may be applied to the testing chamber 172 such as via a port 173. The first deformable component 158 and the second deformable component 160 may respectively bulge out or in when subjected to external pressure or vacuum. For testing, the medical device 112 may be placed in the testing chamber 172 to which pressure or vacuum may be applied. Inside the testing chamber 172 may be the first external deformation sensor 168 and the second external deformation sensor 170 which may respectively be displacement sensors 174. The displacement sensors 174 may be configured to scan surfaces of the first deformable component 158 and the second deformable component 160. The displacement may be measured with a tactile (inductive, incremental, etc.) or non-contact (laser triangulation, confocal, etc.) distance sensor. At the beginning of a test, the displacement sensors 174 may be tared at normal pressure. Then the testing chamber 172 may be brought to a desired positive or negative pressure. A measurement result of the displacement sensors 174 may provide location-selective information about a tightness of a probed tight chamber 116. For calibration, tight and leaky medical devices 112 may be measured and deflections found may be used for referencing. An attributive result (tight/untight) may be available after a fixed measurement time. If deflections of deformable components 122 during a pressure change are detected, information of displacement during time is obtained. A slope of such a curve may provide information about a size of a leakage.

FIG. 1B shows exemplary measurement results. Thereby, a displacement d of the deformable component 122 is shown in dependency of a measurement time t. The solid line shows measurement results corresponding to a tight chamber. The dashed line shows measurement results corresponding to a slightly leaky chamber. The dotted line shows measurement results corresponding to a strongly leaky chamber.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 kit

112 medical device

114 housing

116 tight chamber

118 interior lumen

120 surrounding environment

122 deformable component

124 invasive portion

126 analyte sensor

128 insertion component

130 sterility cap

132 detachable sterility cap

134 axis

136 insertion cannula

138 closed end

140 open end

142 tip

143 holder

144 electronics unit

146 electronics component

148 electronics unit housing

150 first housing

152 first tight chamber

154 second housing

156 second tight chamber

158 first deformable component

160 second deformable component

162 wall

164 wall

166 external deformation sensor

168 first external deformation sensor

170 second external deformation sensor

172 testing chamber

173 port

174 displacement sensor

176 movable component

178 cap

180 invasive portion compartment

182 detachable lower cap

184 detachable upper cap

186 intermediate component

188 sealing element

200 arrow

202 sensor

What is claimed is:

1. A medical device, comprising:
an invasive portion;
a housing;
a tight chamber surrounded by the housing and having an interior lumen, the tight chamber configured for sustaining a pressure difference between the interior lumen and a surrounding environment; and
the housing comprising a deformable component, wherein a state of deformation of the deformable component provides a measurable indication of the pressure difference between the interior lumen and the surrounding environment.

2. The medical device according to claim 1, further comprising an integrated deformation sensor configured for measuring the state of the deformation of the deformable component.

3. The medical device according to claim 1, wherein the deformable component comprises a wall of the housing.

4. The medical device according to claim 1, wherein the deformable component forms a part of the housing that at least partially surrounds the tight chamber.

5. The medical device according to claim 1, wherein the deformable component has a predefined material strength.

6. The medical device according to claim 1, wherein the deformable component comprises at least one material having a tensile strength of 40 MPa to 180 MPa.

7. The medical device according to claim 1, wherein the deformable component has a thickness of 0.1 mm to 1.0 mm.

8. The medical device according to claim 1, wherein the deformable component is made of at least one material selected from the group consisting of acrylonitrile butadiene styrene (ABS), polypropylene, polystyrene, polyamide, polycarbonate, polyethylene, polymethyl methacrylate (PMMA), polyoxymethylene (POM), thermoplastic elastomers (TPE) and silicone.

9. The medical device according to claim 1, wherein the invasive portion is selected from the group consisting of at least one analyte sensor for detecting at least one analyte in a body fluid of a user, at least one insertion component, at least one infusion cannula, and at least one stimulating electrode.

10. The medical device according to claim 1, wherein the invasive portion is at least partially received in the tight chamber.

11. The medical device according to claim 1, further comprising a sterility cap at least partially surrounding the invasive portion, wherein the deformable component forms part of a wall of the sterility cap.

12. The medical device according to claim 1, further comprising an electronics unit having at least one electronics component, wherein the electronics unit further comprises an electronics unit housing, wherein the electronics component is received in the electronics unit housing, wherein the housing is the electronics unit housing, wherein the electronics component is received in the tight chamber and wherein the deformable component forms part of a wall of the electronics unit housing.

13. The medical device according to claim 1, further comprising an electronics unit having at least one electronics component that is received in the tight chamber.

14. The medical device according to claim 13, wherein the deformable component forms part of a wall of the housing.

15. The medical device according to claim 1, wherein the medical device is selected from the group consisting of a medication device for delivering at least one therapeutical medical fluid to a user and a device for detecting at least one analyte in a body fluid of the user.

16. A kit, comprising:
the medical device according to claim 1; and
an external deformation sensor configured for measuring the state of the deformation of the deformable component.

17. A method for monitoring the integrity of a housing of a medical device, comprising:
a) providing a medical device according to claim 1;
b) placing the medical device into a testing chamber and applying a test pressure to the testing chamber;
c) detecting the state of deformation of the deformable component, preferably over time; and
d) evaluating the integrity of the housing of the medical device based on the state of deformation of the deformable component.
